(19) United States
(12) Patent Application Publication
(10) Pub. No.: US 2014/0200515 A1
(43) Pub. Date: Jul. 17, 2014

(54) MEDICAL MARKING APPARATUS AND METHOD OF USE

(71) Applicant: Research Institute at Nationwide Children's Hospital, Columbus, OH (US)

(72) Inventors: AMBER M. Patterson, Westerville, OH (US); Susan J. Bolton, New Albany, OH (US); Gayla J. Rogers, Pickerington, OH (US)

(73) Assignee: Research Institute at Nationwide Children's Hospital, Columbus, OH (US)

(21) Appl. No.: 14/155,626

(22) Filed: Jan. 15, 2014

Related U.S. Application Data
(60) Provisional application No. 61/752,671, filed on Jan. 15, 2013.

(51) Int. Cl. A61B 19/00; A61B 17/34; A61M 5/42

(52) U.S. Cl. CPC A61B 19/54; A61M 5/427; A61B 17/3403
USPC 604/116

(57) ABSTRACT

A medical marking apparatus and method of use for positioning target indicia on a patient. The medical marking apparatus comprises a body member having an upper region and a lower region. The upper region includes a handle for gripping the medical marking apparatus during use. The lower region has a substantially Linear surface projecting a plurality of spaced markers away from said handle.

### CROSS REFERENCES TO RELATED APPLICATIONS

The following application claims priority to co-pending U.S. Provisional Patent Application Ser. No. XXXXX,XXX filed Jan. 15, 2013 entitled MEDICAL MARKING APPARATUS AND METHOD OF USE under attorney docket number NCH-021847 US PRO and U.S. Design Patent Application Ser. No. 29/XXX,XXX filed Jan. 15, 2013 entitled MEDICAL MARKING APPARATUS under attorney docket number NCH-021847 US DES. The above-identified applications are incorporated herein by reference in their entireties for all purposes.

### TECHNICAL FIELD

The present disclosure relates to a medical marking apparatus and method of use and more particularly, a medical marking apparatus for identifying one or more testing or treatment locations for medical procedures performed on a patient.

### BACKGROUND

Various medical testing procedures require penetrating the patient's skin with a medical device such as a short needle-like lance, often to a depth less than the subcutaneous tissue layer. The lance is typical carries a testing agent that may result in the reaction to the skin cells surrounding the penetrated region defined as the testing area

The testing area is typically observed once the patient is exposed to the agent by the medical device for any reaction to the skin cells. if a reaction to the agent occurs, traumatized skin cells on the patient at the testing area become inflamed and are often no larger than two or three centimeters. For some patients, it is possible for the skin reaction to take fifteen to twenty minutes.

Conventional allergy testing often referred to as a prick test similarly uses a lance carrying an allergen that penetrates a testing area in order to release the allergen into the patient's skin. The testing area is then monitored for a period of time to see if any traumatized skin cells are generated, thus providing a visible allergic reaction.

### SUMMARY

One example embodiment includes a medical marking apparatus and method of use for positioning target indicia on a patient. The medical marking apparatus comprises a body member having an upper region and a lower region. The upper region includes a handle for gripping the medical marking apparatus during use. The lower region has a substantially linear surface projecting a plurality of spaced markers away from the handle.

Another example embodiment includes a method of performing a medical procedure on a patient. The method comprises the steps of grasping a handle located on a medical marking apparatus and placing marking fluid on a plurality of markers located on an underside of the handle of the medical marking apparatus. The method further comprises transferring at least a portion of the marking fluid from the plurality of markers to a patient in a target area by contacting the plurality of markers with the patient's skin in the target area to form a plurality of target indicia.

### BRIEF DESCRIPTION OF THE DRAWINGS

The foregoing and other features and advantages of the present disclosure will become apparent to one skilled in the art to which the present invention relates upon consideration of the following description of the invention with reference to the accompanying drawings, wherein like reference numerals refer to like parts unless described otherwise throughout the drawings and in which:

FIG. 1 is an upper perspective view of a medical marking apparatus constructed in accordance with one example embodiment of the present disclosure;

FIG. 2 is a lower perspective view of FIG. 1;

FIG. 3 is a front elevation view of FIG. 1;

FIG. 4 is a rear elevation view of FIG. 1;

FIG. 5 is a top plan view of FIG. 1;

FIG. 6 is a bottom plan view of FIG. 1;

FIG. 7 is a first side elevation view of FIG. 1

FIG. 8 is a second elevation view of FIG. 1;

FIG. 9 is an upper perspective view of a medical marking apparatus constructed in accordance with a second embodiment of the present disclosure;

FIG. 10A is a lower perspective view of FIG. 9;

FIG. 10B is a lower perspective view of FIG. 9 constructed in accordance with another example embodiment of the present disclosure;

FIG. 11 is a front elevation view of FIG. 9;

FIG. 12 is a rear elevation view of FIG. 9;

FIG. 13 is a top plan view of FIG. 9;

FIG. 14 is a bottom plan view of FIG. 9;

FIG. 15 is a first side elevation view of FIG. 9;

FIG. 16 is a second elevation view of FIGS. 9; and

FIG. 17 is a perspective view of the medical marking apparatus in use on a patient in accordance with the example embodiments of the present disclosure;

FIG. 18 is a perspective view of a patient's arm that has been treated with a medical marking apparatus in accordance with the example embodiment of FIGS. 9 and 10 of the present disclosure; and

FIG. 19 is a perspective view of a patient's arm that has been treated with a medical marking apparatus in accordance with the example embodiment of FIGS. 1 and 2 of the present disclosure.

### DETAILED DESCRIPTION

Referring now to the figures generally wherein like numbered features shown therein refer to like elements throughout unless otherwise noted. The present disclosure relates to a medical marking apparatus and method of use, and more particularly, a medical marking apparatus for identifying one or more testing or treatment locations for medical procedures performed on a patient.

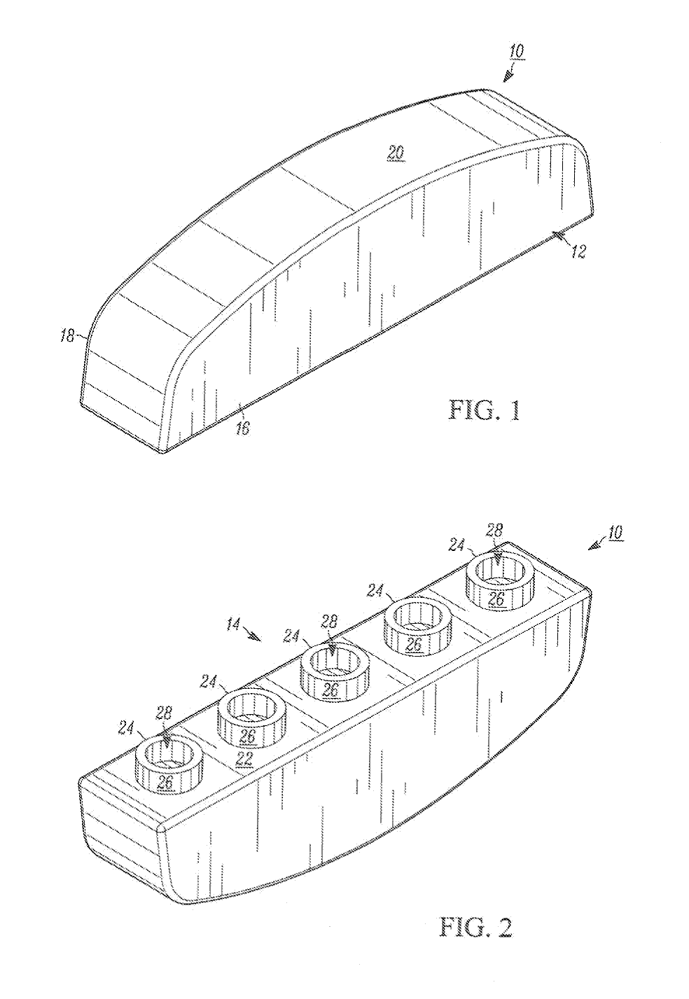

FIG. 1 illustrates a perspective view of a medical marking apparatus 10 constructed in accordance with one example embodiment of the present disclosure. The medical marking apparatus 10 comprises an upper region 12 and a lower region 14 (see, for example FIG. 2). The upper region 12 forms an arcuately shaped handle having first and second sides 16 and 18, respectively divided by a spine or main body 20.

The lower region 14 comprises a linear surface 22 extending along the length of the body 20. The lower region 14 further comprises a plurality of markers 24 projecting out and away from the linear surface 22. In the illustrated example embodiment, five markers 24 are shown axially aligned symmetrically about an axis X in FIG. 6. However, it should be appreciated that apparatus 10 includes any number of markers 24 without departing from the spirit and scope of the present disclosure.

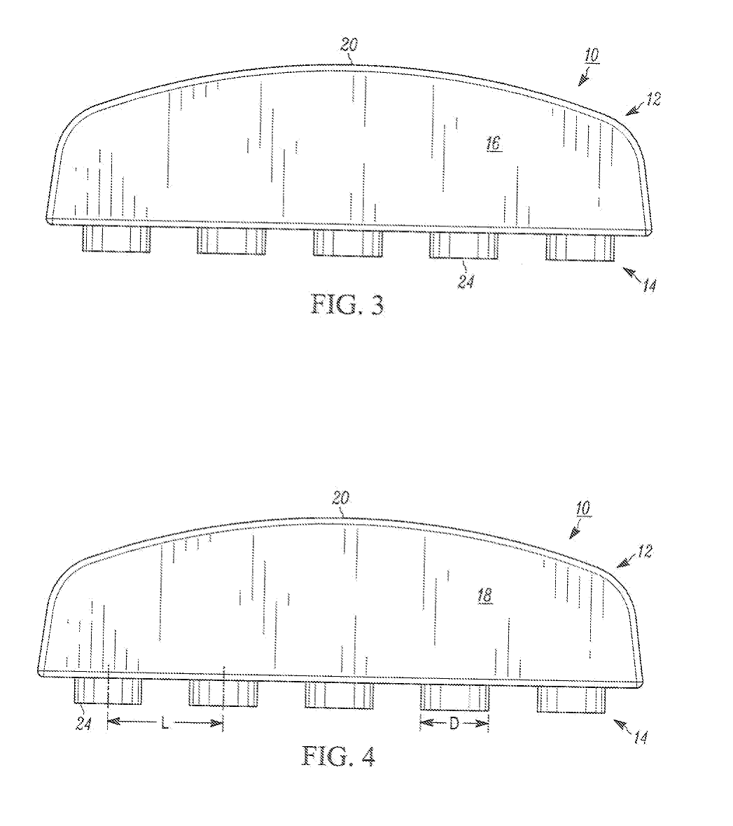

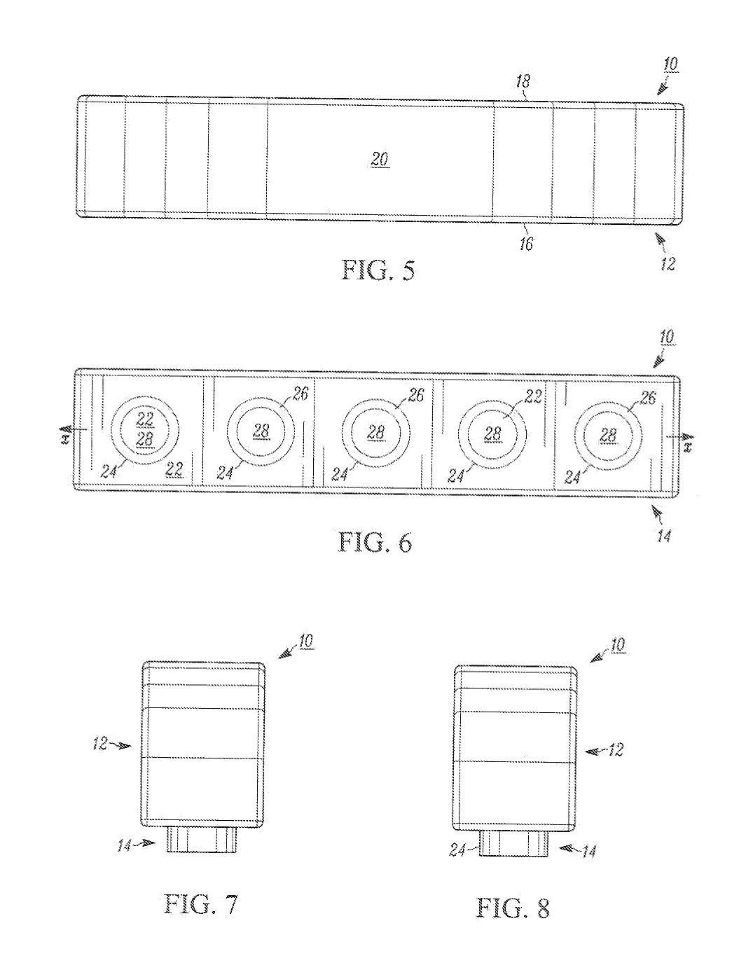

In the illustrated example embodiment of FIGS. 1-8, the markers 24 are cylindrically shaped and molded into and project from the linear surface 22 of the body 20. Each of the cylindrical markers 24 comprise an annular wall 26 having an inner and outer surface forming a cavity or open area 28, as best seen in FIG. 2. The cavity 28 has an annular shape defined by the annular wall 26 about its perimeter a base formed by the linear surface 22.

In one example embodiment, the cylindrical markers 24 have a diameter of approximately 2 cm (indicated by reference character D) and the diametrical centers of the markers are spaced by approximately 3 cm (indicated by reference character L). Stated another way, the distance between each cylindrical marker 24 in one-example embodiment is 1 cm, leaving 3 cm between centers on a 2 cm diameter marker.

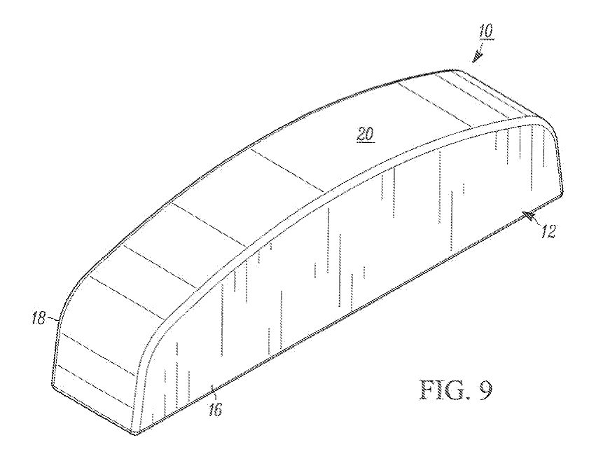

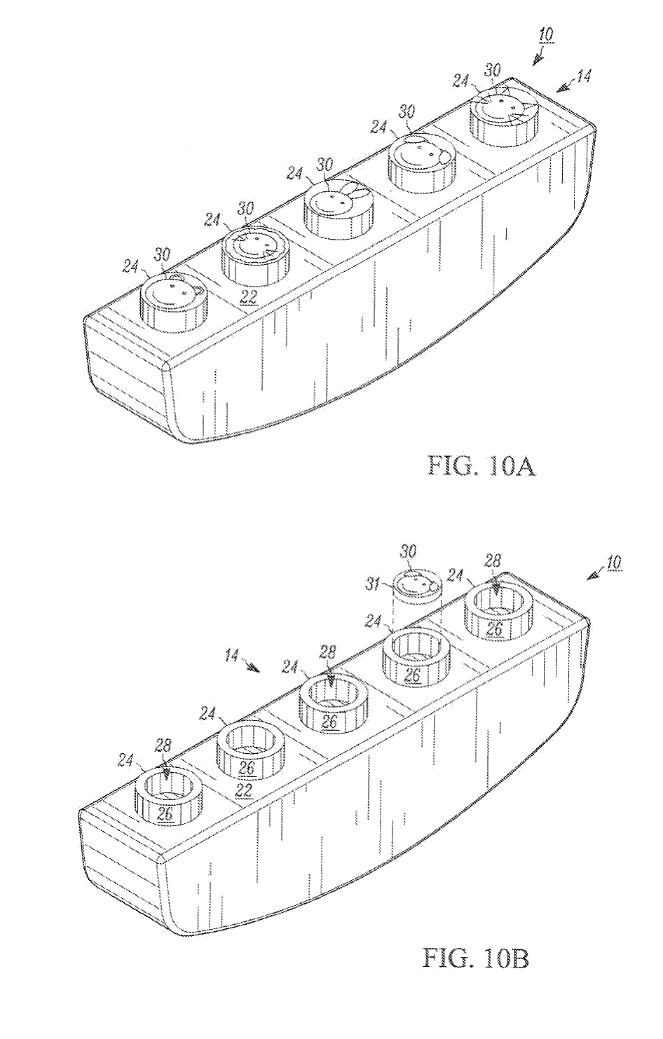

Referring now to FIG. 9 is an upper perspective view of a medical marking apparatus 10 constructed in accordance with a second example embodiment of the present disclosure. The medical marking apparatus 10 comprises an upper region 12 and a lower region 14 (see, for example FIGS. 10A and 10B). The upper region 12 forms an arcuately shaped handle having first and second sides 16 and 18, respectively divided by a some or main body 20.

The lower region 14 comprises a linear surface 22 extending along the length of the body 20. The lower region 14 further comprises a plurality of markers 24 projecting out and away from the linear surface 22. In the illustrated example embodiment, five markers 24 are shown axially aligned symmetrically about an axis X shown in FIG. 14. However, it should be appreciated that apparatus 10 includes any number of markers 24 without departing from the spirit and scope of the present disclosure.

In the illustrated example embodiment of FIGS. 9, 10A, and 16, the markers 24 are solid cylinders and molded into and project from the linear surface 22 of the body 20. Each of the cylindrical markers 24 further comprise an outer surface 29 supporting a character 30. In the illustrated example embodiment, the characters 30 are molded into the cylindrical markers 24 and project outward from the outer surface 29.

As best seen in FIG. 14, the characters 30 comprise a plurality of different animal shapes, namely a bear, lion, rabbit, puppy, and cat. It should however be appreciated that the characters 30 could be any number of animals, symbols, and/or shapes without departing from the spirit and scope of the present disclosure.

In the alternative example embodiment of FIG. 10B, the markers 24 are cylindrically shaped as in the embodiment of FIG. 1, but designed to receive caps 31 having an assortment of different characters 30 that snap into the cavity or open areas 28 on each of the markers. The caps 31 are selectively removable, allowing different character shapes to be added or changed to the preference of the medical practitioner. In the illustrated example embodiment, the caps 31 are made from plastic, but could be made from other materials without departing from the spirit and scope of the present disclosure.

In one example embodiment, the cylindrical markers 24 and more specifically the characters 30 have a diameter of approximately 2 cm (indicated by reference character D) and the diametrical centers are spaced by approximately 3 cm (indicated by reference character L). Stated another way, the distance between each cylindrical marker 24 in one-example embodiment is 1 cm, leaving 3 cm between centers on a 2 cm diameter marker.

In the illustrated example embodiments, the medical marking apparatus 10 is constructed from a polymer such a polyethylene, polypropylene, polystyrene, molded plastic, and the like. Such materials allow the apparatus 10 to be manufactured very economically and are more suitable for disposal after a single use on a single patient.

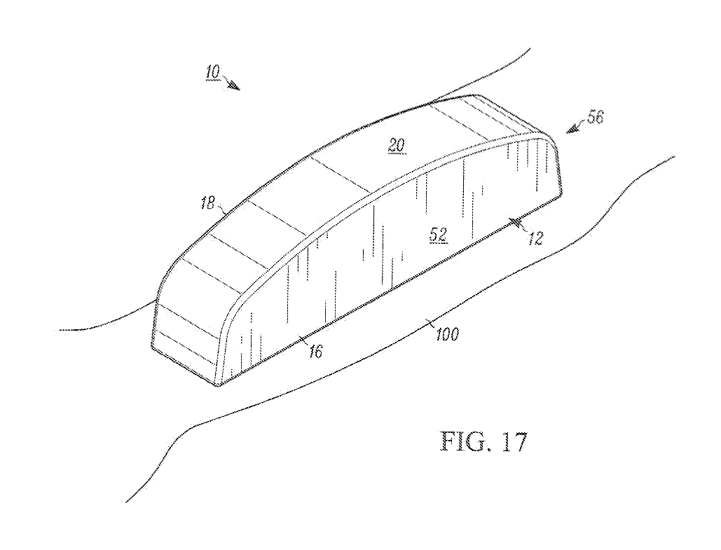

Referring now to FIG. 17 is a perspective view of the medical marking apparatus 10 in use on a patient 100 in accordance with the example embodiments of the present disclosure. FIG. 17 illustrates the apparatus 10 being placed on a patient's arm, but could equally be positioned on any other part of the patient's body suitable for testing or medical treatment, such as, for example the patient's leg or back.

During use, the medical marking apparatus 10 is grasped by a medical practitioner, such as a nurse or doctor in a handle area 52 formed in the upper region 12. The plurality of markers 24 of the lower region 14 are coated with marking fluid 54, such as non-toxic ink. This can be achieved by submerging the plurality of markers 24 into an inkpad or sponge coated with the marking fluid 54. Alternatively, the marking fluid 54 is painted with a brush or sprayed onto the markers 24. Before the marking fluid 54 dries, at least a portion of the fluid is transferred from the plurality of markers 24 to the patient in a target area 56, as illustrated in the example embodiment of FIG. 17. In this example embodiment, the target area 56 is the arm, but could be any part of the body that is subject to treatment or testing.

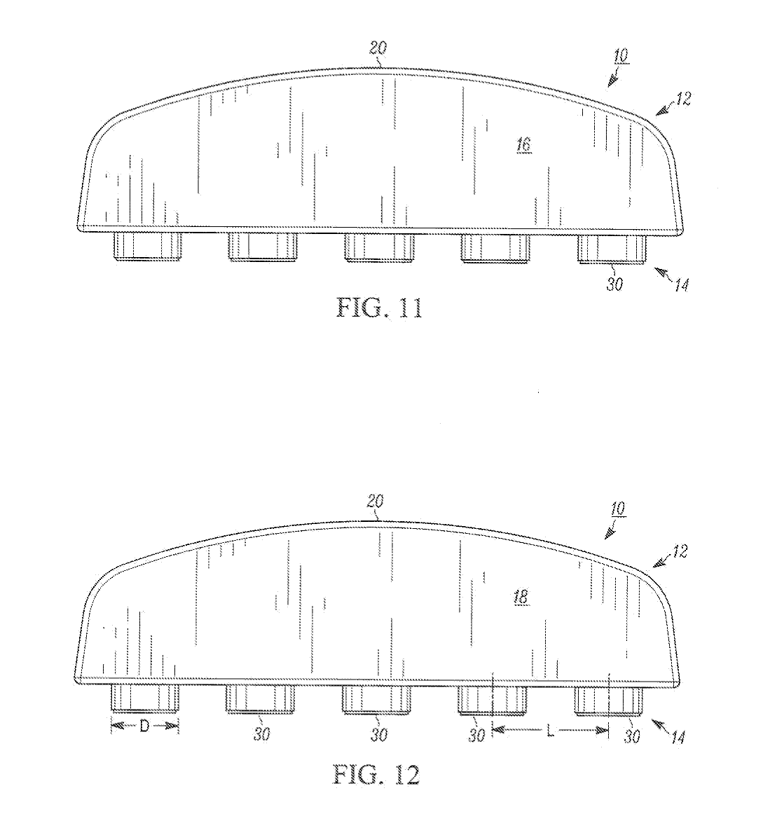

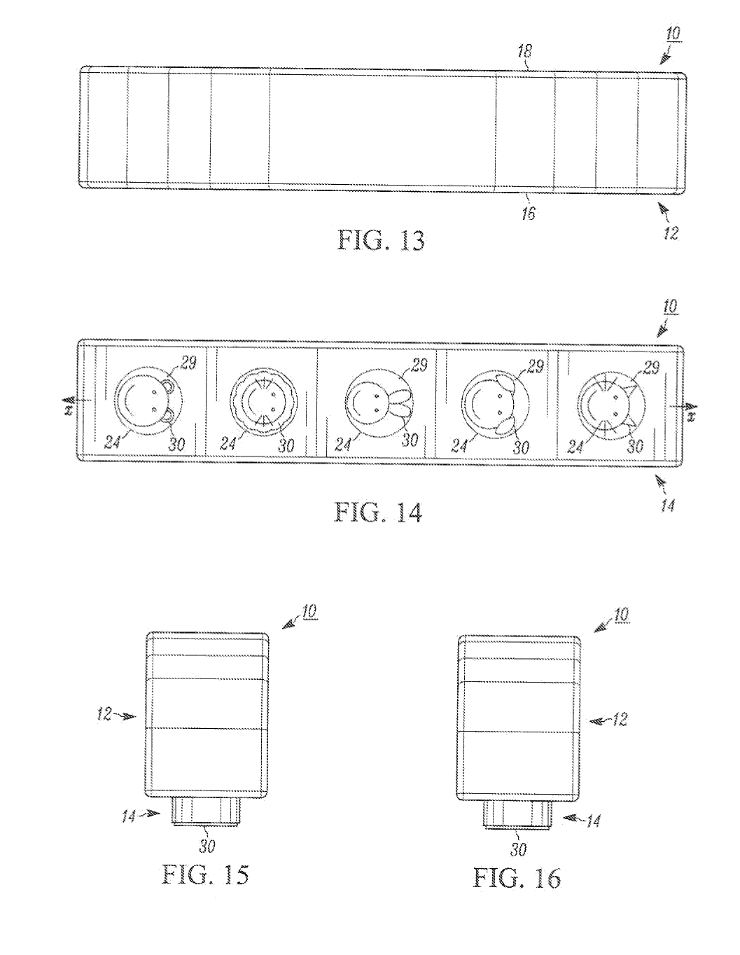

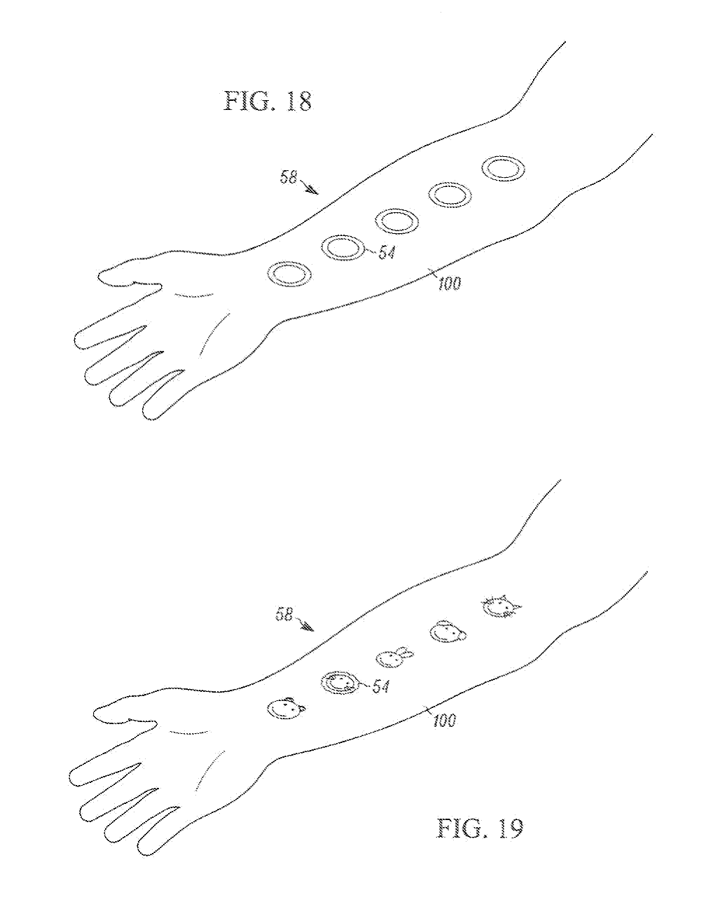

Once contact is made between the patient's skin and plurality of markers 24, the apparatus 10 is removed and a plurality of target indicia 58 is formed on the patient's arm corresponding to each of the markers as illustrated in FIGS. 18 and 19. In particular, the patient's arm in FIG. 18 has target indicia 58 corresponding to the apparatus 10 shown in FIGS. 1-8, while the arm shown in FIG. 19 has target indicia 58 corresponding to the apparatus illustrated in FIGS. 9-16.

Once the target indicia 58 are applied to the surface of the patient's skin, the medical practitioner can advantageously track testing or treatment by monitoring the targets after subsequent procedures is performed within the targets on the patient. For example, the practitioner may penetrate the patient's skin with a lance with a different allergen in each of the target indicia 58. Without the target indicia 58, such tests or treatments would leave the practitioner guessing as to the location, especially when multiple tests or treatments are required. Skin cells can then be easily monitored within each target indicia 58 for irritation without uncertainty as to where the lance occurred, the type of allergen used and at what location, where the testing or treatment started, etc.

Yet another benefit to the example embodiment of FIGS. 9-16 is the therapeutic reaction pediatric patients experience when exposed to various cartoon characters 30 when placed upon their skin. Such characters 30 keep the children occupied and may even induce their participation in the medical procedure. For example, if an allergen is placed by a lance in the nose area of the target indicia 30, a reaction or nose should appear. Stated another way, the child may assist by watching for a nose to grow on the animal character 30, such as the bear or rabbit if a reaction occurs. Such cooperation with the pediatric patient would improve the behavior or help relax child patient.

What have been described above are examples of the present invention. It is, of course, not possible to describe every conceivable combination of components or methodologies for purposes of describing the present invention, but one of ordinary in the art will recognize that many further combinations and permutations of the present invention are possible. Accordingly, the present invention is intended to embrace all such alterations, modifications, and variations that fall within the spirit and scope of the appended claims.

What is claimed is:

1. A medical marking apparatus for positioning target indicia on a patient, the medical marking apparatus comprising:
a body member having an upper region and a lower region, the upper region forming a handle for gripping the medical marking apparatus during use;
said lower region having a substantially linear surface projecting a plurality of spaced markers away from said handle.

2. The medical marking apparatus of claim 1 wherein said body member is a single molded piece comprising both the upper region and lower region.

3. The medical marking apparatus of claim 1 wherein said body member is a single molded piece formed from a polymeric material.

4. The medical marking apparatus of claim 1 wherein said plurality of markers are cylindrically shaped projections.

5. The medical marking apparatus of claim 4 wherein said plurality of markers comprise an annular cavity within said cylindrically shaped projections for positioning cylindrically shaped target indicia on a patient.

6. The medical marking apparatus of claim 4 wherein said plurality of markers comprise a character for positioning character target indicia on a patient.

7. The medical marking apparatus of claim 6 wherein each of said plurality of markers further comprise a different character.

8. A method of performing a medical procedure on a patient, the method comprising the steps of:
grasping a handle located on a medical marking apparatus;
placing a marking fluid on a plurality of markers located on the underside of the handle of the medical marking apparatus;
transferring at least a portion of the marking fluid from the plurality of markers to a patient in a target area by contacting the plurality of markers with the patient's skin in the target area to form a plurality of target indicia.

9. The method of claim 8 further comprising the step of treating a patient by penetrating the target indicia with a medical device.

10. The method of claim 8 further comprising the step of diagnostically testing a patient by penetrating the target indicia with a medical device.
